(19) United States
(12) Patent Application Publication
(10) Pub. No.: US 2003/0068786 A1
(43) Pub. Date: Apr. 10, 2003

(54) Process for the production of egg yolk antibodies for organochlorine pesticides

(76) Inventors: Bangalore Eahwar Amita Rani, Mysore (IN); Akmal Pasha, Mysore (IN); Nitturu Gopalakrishna Karanth, Mysore (IN); Raja Rao Jagannath Rao, Mysore (IN); Putte Gowda, Mysore (IN)

Correspondence Address: FOLEY AND LARDNER, SUITE 500, 3000 K STREET NW, WASHINGTON, DC, 20007, US

(21) Appl. No.: 09/973,199

(22) Filed: Oct. 10, 2001

(51) Int. Cl. A01K 67/027; C12P 21/04

(52) U.S. Cl. 435/70.21; 435/70.3; 800/19

(57) ABSTRACT

The present invention provides relates to a process to produce egg yolk antibodies binding to small molecule organochlorine pesticides, by periodic immunization of the poultry birds with a desired hapten-protein conjugate in the breast muscle. Antibodies are harvested from egg yolk after five weeks interval.

### FIELD OF INVENTION

The present invention relates to the production of egg yolk antibodies binding to small molecule organochlorine pesticides. The present invention also relates to periodic immunization of the poultry birds with a desired hapten-protein conjugate in the breast muscle.

### BACKGROUND OF THE INVENTION

The organochlorine pesticides are large class of insecticides that generally accumulate in the tissues and are difficult to degrade as they have very long half-life periods.

Organochlorine insecticides are used widely for treatment of crops such as cereals, fruits, vegetables, legumes, cotton, coffee and tea, forest trees, sanitation and domestic mosquito/malaria control programs.

Small organic molecules such as pesticides are not by themselves immunogenic, but can be made immunogenic following chemical conjugation to a suitable carrier such as a protein. The site and nature of conjugation and carrier protein can influence the specificity and affinity of antibodies produced, in a manner not fully able to be predicted by those skilled in the art. For example, preparation of DDA-GABA (a compound closely related to the hapten used in the present invention coupled with-bovine serum albumin) did not yield conjugates producing antisera with useful reaction with—DDT. (Amitarani, B. E., Akmal Pasha and Karanth N. G. K. (1998, unpublished data).

In recent years, techniques of producing antibodies (polyclonal and monoclonal) have been developed which make it possible to obtain homogeneous, highly specific antibodies. Generally, polyclonal antibodies are used extensively in diagnostics industry. Most commonly they are raised in mammals such as rabbits, mice, rats, horses and goats. This form of antibody production has several disadvantages—large mammals are expensive to maintain, while small mammals yield small quantities of antibody. In addition there is a requirement for periodic drawing of blood from the animals (Deignan et al. 2000)). The amount of IgG (antibody) obtained is usually between 3-8 mg/ml of serum. The method also involves bleeding of the rabbit several times to obtain the antibodies, as the titer is highest only between the 8^{th}-10^{th} day after 2-3 boosters. Monoclonal antibodies are produced by immunizing an animal with a protein, obtaining antibody-producing cells from the animal, and fusing the antibody-producing cells with strains of myeloma cells, i.e., tumor cells, to produce hybridomas, which are isolated and cultured as monoclones. The monoclonal hybridomas may either be cultured in vitro or from the cells, ascitic fluid, or serum of a tumor-bearing host animal. Since each antibody-producing cell generates a single unique antibody, each monoclonal culture of hybridomas produces a homogeneous antibody. Not all of the hybridoma clones, which result from fusing myeloma cells with antibody-producing cells, are specific for the desired pesticide (or for functional groups upon the pesticide characteristic of that class of molecules), since many of the hybridomas will make antibodies, which the inoculated animal has produced to react with other foreign substances. Even antibodies against the subject antigen will differ from clone to clone, since antibodies produced by different cells may react with different antigenic determinants of the same molecule. From each clone, therefore, it is necessary to obtain the resulting antibody and test its reactivity with the subject pesticides and to test its specificity by determining which particular organochlorine pesticide it recognizes. Further, only certain antibodies or antisera function in specific immunoassay formats or configurations.

U.S. Pat. Nos. 4,387,272 and 4,550,019 to Polson; and Losch, U claimed production of hen egg yolk antibodies and has been used in a number of applications for passive transfer of immunity.

U.S. Pat. No. 4,748,018 to Stolle, et al. discloses a method of passive immunization against bacterial infection comprising a preliminary development of tolerance to HEY by repeated oral ingestion of egg yolk, followed by parenteral injection of HEY antibody to a selected bacterial antigen.

U.S. Pat. No. 5,080,895 to Tokoro discloses prevention of E. Coli diarrhoea in newborn piglets by oral administration of anti-bacterial hen egg yolk antibodies.

Reference is made to Hamada, S., Infection and Immunity 59(11): 4161-4167 (1991); and Otake, S., J. Dental Research 70(3): 162-166 (1991) reproduced the results of Beck in protecting rats against dental caries by means of passive immunization with orally administered hen egg yolk antibodies against S. mutans.

Reference is made to Bartz, C. et al., J. Infectious Disease 142(3): 439-441 (1980) prevented murine rotaviral infection in mice by the oral administration of the water-soluble fraction of the eggs of immunized hens

Reference is made to Yokoyama, H., et al. Infection and Immunity 60(3): 998-1007 (1992) succeeded in passively protecting neonatal piglets from fatal enterotoxigenic E. coli infection by oral administration of a crude yolk immunoglobulin fraction from the eggs of immunized hens.

Reference is made to the animal studies of Yolken, R. et al., Pediatrics 81(2): 291-295 (1988); and Journal Clint. Immunol. 10(6): 80S-87S (1990), proposed the oral administration of antiviral HEY immunoglobulin for the prevention and treatment of enteric infections, including rotaviral infection in humans. Methods and formulations for the oral administration of immune globulin are known (U.S. Pat. No. 4,477,432 to Hardie). However, there are no reports on the production of egg yolk antibodies for a small molecule such as a pesticide.

### OBJECTS OF THE PRESENT INVENTION

The main object of the present invention, is to provide antibodies from the egg yolk of hyper immunized hens (HEY antibody) for organochlorine insecticides.

Another object of the present invention is to provide continuos supply of large quantities of consistent, high titer and specific antibodies that can be easily collected and stored.

Yet another object of the present invention is to provide a non-invasive, hence no need to bleed the animal.

Still another object of the present invention, is to provide antibodies that are equally or more sensitive to both polyclonal and monoclonal antibodies produced using mammals.

### SUMMARY OF THE PRESENT INVENTION

The present invention provides relates to a process to produce egg yolk antibodies binding to small molecule organochlorine pesticides, by periodic immunization of the poultry birds with a desired hapten-protein conjugate in the breast muscle. Antibodies are harvested from egg yolk after five weeks interval.

### DESCRIPTION OF THE INVENTION

Accordingly, the present invention provides a process for the production of egg yolk antibodies binding to small molecule organochlorine pesticides, the said process comprising the steps of:

(a) selecting suitable from poultry birds;

(b) immunizing the poultry birds with known complete adjuvant, each ml of said adjuvant comprising heat killed and dried 1 mg of Mycobacterium tuberculosis (H37Ra, ATCC 25177), 0.85 ml paraffin and 0.15 ml mannide monooleate;

(c) immunizing the birds with 1000 μg conjugate of selected from DDT-OH hapten, Octachloro cyclic hapten, 2,4,5 trichlorophoxyacetic acid β-alanine in breast muscle;

(d) immunizing the birds again with the hapten-protein conjugate as given in step (c) with 500 μg of desired hapten conjugate;

(e) immunizing the birds with hapten-protein conjugate at the intervals of two, three and five weeks;

(f) immunizing the birds thereafter with hapten-protein conjugate at five weeks intervals as long as the bird lays eggs;

(g) harvesting antibodies from the egg yolk of the birds.

An embodiment of the present invention, wherein the desired hapten-protein conjugates having binding properties to DDT, Endosulphan and HCH.

Another embodiment of the present invention, wherein the production of hapten-protein conjugate namely DDT-OH binding to DDT, is as follows:

(a) succinylating 2,2,-Bis(4-chlorophenyl)-1,1,1-trichloroethanol overnight, using excess succinic anhydride in pyridine to obtain N-hydroxy succunimide;

(b) reacting N-hydroxy succinimide 183.5 mg., 0.5 mmol in dichloromethane in the presence of dicyclohexylurea and dimethylaminopyridine catalyst in the ratio 1:1:1:1.2 (hapten:NHS:DCC:DMAP) to convert into N-Hydroxy succinimide active ester; and

(c) obtaining active ester of DDT-OH hapten for use in conjugation by isolating dicyclohexylurea and evaporating dichloromethane.

Yet another embodiment of the present invention, wherein the production of hapten-protein conjugate namely octachloro cyclic hapten binding to Endosulphan, is as follows:

(a) dissolving about 3.73 g Heptachlor in 0.1 mol glacial acetic acid by warming;

(b) dissolving 1.085 g Tert-Butyl hypochlorite, in 0.1 mmol glacial acetic acid and adding to the first solution as obtained in step(a);

(c) refluxing the mixture on a water-bath for 1 hour;

(d) separating fine crystals of acetyl-chloro derivative of heptachlor;

(e) washing the crystals with acetone and drying with air;

(f) obtaining the crystalline product in a yield of about 3.02 g, m.p. 238 C. 1.09 g

(g) treating the product to get the pre-hapten 1,3,4,5,6,7,8,8-Octachloro-2-hydroxy-4,7-methano-3a, and 4,7,7a-tetrahydroindane;

(h) dissolving the pre hapten in dichloromethane by adding N-hydroxysuccinimide and cooling the mixture to 0° C.;

(i) adding dicyclohexylcarbodiimide followed by dimethylaminopyridine;

(j) stirring the mixture overnight; and

(k) filtering off dichloromethane and evaporating dichloromethane to obtain the active ester of endosulphan.

Still another embodiment of the present invention, wherein the production of conjugate 2,4,5-Trichloro phenoxy acetic acid β-alanine Trichloro benzene (TCB) hapten binding to Hexachloro hexane, is as follows:

(a) adding of β-alanine spacer arm to 2,4,5 Trichlorophenoxyacetic acid by suspending 10 mM, 2.55 g of 2,4,5 Trichlorophenoxyacetic acid in 5.95 ml thionyl chloride (9 50 mmol);

(b) refluxing for 1 hour and removing unreacted thionyl chloride by evaporation;

(c) stirring the product with β-alanine 9 mmol, 0.66 g in 7.4 ml of 1M NaOH at 0° C.;

(d) warming the product for over 16 hours at room temperature;

(e) isolating the resulting acid by acidification;

(f) partitioning into ethyl acetate;

(g) washing with water and brine;

(h) giving an yield of crude product hapten containing 2,4,5-Trichlorophenoxyacetic acid (2,4,5-T) as impurity;

(i) dissolving the impurity in acetone to obtain colorless flakes of the Trichlorobenzene(TCB) hapten;

(j) filtering and washing the colourless TCB hapten with acetone and drying in air;

(k) using silica gel precoated aluminum plates and a mixture of chloroform and methanol 85:15 as eluent showed a single spot in TLC analysis Rf- 0.45 detected by spraying with 2% o-tolidine in acetone and exposure to Uv light/sunlight, at a melting range of 169-170° C.

(l) synthesizing the active ester of hapten 2,4,5-T-β-alanine at melting range of 102-104° C. by dissolving in dichloromethane.

(m) adding N-hydroxysuccinimide and the mixture is cooled in an ice-bath;

(n) adding Dimethylsulphoxide(DMSO) dropwise to the mixture until the hapten is dissolved;

(o) adding Dicyclohexylcarbodiimide to the mixture followed by adding dimethylaminopyridine catalyst;

(p) stirring the mixture overnight and the temperature slowly raised to the room temperature;

(q) filtering and evaporating acetone; and

(r) separating the active ester as a colorless solid.

Yet another embodiment of the present invention, wherein harvesting of antibodies is conducted as follows:

(a) obtaining eggyolk without rupturing the yolk;

(b) adding 100 ml of Tris buffer saline for every 10 ml of yolk;

(c) removing the precipitate by centrifugation;

(d) adding to the supernatant the precipitating solution of magnesium chloride and phosphotungstic acid for centrifuging;

(e) discarding the pellet;

(f) adding to the supernatant a water soluble protein fraction 12% polyethylene glycol;

(g) incubating for 10 minutes and then centrifuging again;

(h) precipitating out the antibody;

(i) adding 10 ml of 10 mM phosphate buffer to dissolve the precipitate;

(j) cooling the antibody solution 0° Cl;

(k) adding 10 ml of pre-cooled ethanol;

(l) centrifuging the solution at 4° C. and dissolving the sediment in 10 mM phosphate buffer; and

(m) dialyzing against phosphate buffer for 24 h at 4° C. to obtain the yield of antibodies.

Still another embodiment of the present invention, wherein harvesting of antibodies can also be conducted as follows:

(a) obtaining the egg yolk from the eggshell without rupturing the yolk membrane;

(b) adding for every 10 ml of yolk, 10 ml of distilled water;

(c) adding about 0.15% of kappa-carragenanin and left to stir for 30 minutes at room temperature;

(d) filtering and centrifuging the solution at for 15 minutes;

(e) passing through the DEAE-sephacel column prepared with 20 mM phosphate buffer pH 8.0;

(f) eluting with 0.2 M phosphate buffer pH 8.0;

(g) collecting the eluate and the absorbance read at 280 nm; and

(h) pooling and storing the peak fractions containing the antibody at 4° C.

Yet another embodiment of the present invention, wherein the lipid from egg yolk is precipitated out twice using the precipitating solution of phosphotungstic acid and magnesium chloride and centrifuged obtaining the antibody yield up to 75% from supernatant.

Still another embodiment of the present invention, wherein pH of the water soluble protein fraction obtained after the removal of the lipids is adjusted to pH 5.0 to further precipitate out the antibodies for obtaining a yield of 80-90%.

Yet another embodiment of the present invention, wherein the yield of antibody is to the extent of 73%.

Still another embodiment of the present invention, wherein the hyper immune eggs are collected daily and stored 40° C. until further use.

Yet another embodiment of the present invention, wherein commencing the production of the antibody from 7^{th} day after the immunization and continued for 60 days.

Still another embodiment of the present invention, wherein the titer of the antibody produced is 165-225 mg/ml.

Yet another embodiment of the present invention, wherein production of the egg yolk antibody is more/equally sensitive to the polyclonal/monoclonal antibodies produced using mammals.

Still another embodiment of the present invention, wherein production of the egg yolk antibodies relates to small molecules of pesticides.

Yet another embodiment of the present invention, wherein the production of egg yolk antibodies binding to the following organochlorine insecticides selected from:

a) (DDT—1,1′-(2,2,2-Trichloroethylene)bis(4-chlorobenzene),

b) HCH—1,2,3,4,5,6-hexachlorohexane

c) ENDOSULPHAN—6,7,8,9,10,10-hexachloro-1,5,5a,6,9,9a, hexahydro-6,9-methano-2,3,4-benzodioxathiepin-3-oxide.)

The following examples are given by way of illustration of the present invention and therefore should not be construed to limit the scope of the present invention.

### EXAMPLES

### Example-1

The egg yolk was carefully removed from the eggshell without rupturing the yolk membrane. The entire albumin adhering to the membrane was washed off. And the egg yolk membrane was ruptured and transferred into a measuring cylinder through a funnel. For every 10 ml of yolk, 100 ml of Tris buffer saline was added. The precipitate formed was removed by centrifugation. To the supernatant the precipitating solution of magnesium chloride and phosphotungstic acid was added and centrifuged again. The pellet discarded and to the supernatant now called the water soluble protein fraction 12% polyethylene glycol was added and incubated for 10 minutes and then centrifuged. The antibody precipitates out. 10 ml of 10 mM phosphate buffer is added and the precipitate dissolved. The antibody solution is then cooled to 0° C. and 10 ml of pre cooled ethanol added. The solution is centrifuged at 4° C. and the sediment is dissolved in 10-mM phosphate buffer and dialyzed in phosphate buffer for 24 h at 4° C. Yield—60%.

### Example-2

The lipid from the egg yolk was precipitated out twice using the precipitating solution of phosphotungstic acid and magnesium chloride from the supernatant and centrifuged. Yield—75%.

### Example-3

The pH of the water soluble protein fraction obtained after the removal of the lipids was checked and adjusted to pH 5.0 in order to further precipitate out the antibodies. Yield—80-90%.

### Example-4

The egg yolk was carefully removed from the eggshell without rupturing the yolk membrane. The entire albumin adhering to the membrane was washed off. And the egg yolk membrane was ruptured and transferred into a measuring cylinder through a funnel. For every 10 ml of yolk, 10 ml of distilled water was added. 0.15% of kappa-carragenanin was added and left to stir for 30 minutes at room temperature. The solution was then filtered and centrifuged at 10,000 g for 15 minutes. The pH of the supernatant solution was set to pH 8.0 and then passed through the DEAE-sephacel column prepared with 20 mM phosphate buffer pH 8.0. The antibody was eluted with 0.2 M phosphate buffer pH 8.0. The eluate was collected and the absorbency read at 280 nm. The peak fractions containing the antibody were pooled and stored at 4° C. Yield—73%.

The efficacy of these antibodies has also been inferred through this invention.

ADVANTAGES

The main advantages of the present invention are:

(a) The use of antibodies from the egg yolks of hyperimmunized hens (HEY antibody) for immunological procedures overcome the limitations associated with the polyclonal and monoclonal antibodies, because the present method provides a continuous supply of large quantities of consistent, high titer specific and sensitive antibody which can be easily collected and stored.

(b) The sensitivity of the assay is equal or better than rabbit antibodies.

(c) The method of immunizing the poultry birds is non-invasive and has a good affinity to the analyte.

The advantages of antibodies produced by chicken over rabbit are further illustrated in the tabular form as provided in the following Table I.

TABLE I

| | | | RABBITS(2-4 |
|---|---|---|---|
| PARAMETERS | | LAYING HENS(4 NOS) | NOS) |
| A. MAINTENANCE COST | | | |
| IN Rs. | | | |
| (a) | Investment for cage | 15,000 | 20,000 |
| (ii) | Price for each animal | 60 | 75 |
| B. ANTIBODY | | 40 mg/ml | 7 mg/ml |
| PRODUCTION | | 2000-2800 mg | 200 mg |
| (i) | Monthly | | |
| (ii) | Amount of specific | up to 10% | 5% |
| | antibody | | |
| C. INFRASTRUCTURE | | Poultry birds | Small animals |
| Area needed | | | |
| D. CHARACTERISTICS | | | |
| i) | Antibody sampling | Non-invasive | Invasive |
| ii) | Purification | Affinity | Affinity |
| iii) | Interference with | No | Yes |
| | rheumatoid factor | | |
| iv) | Activation of | No | Yes |
| | mammalian | | |
| | complement | | |

1. A process for the production of egg yolk antibodies binding to small molecule organo chlorine pesticides, the said process comprising the steps of:
(a) selecting suitable from poultry birds;
(b) immunizing the poultry birds with known complete adjuvant, each ml of said adjuvant comprising heat killed and dried 1 mg of Mycobacterium tuberculosis (H37Ra, ATCC 25177), 0.85 ml paraffin and 0.15 ml mannide monooleate;
(c) immunizing the birds with 1000 μg conjugate selected from DDT-OH hapten, Octachloro cyclic hapten, 2,4,5 trichlorophoxyacetic acid β-alanine in breast muscle;
(d) immunizing the birds again with the hapten-protein conjugate as given in step (c) with 500 μg of desired hapten conjugate;
(e) immunizing the birds with hapten-protein conjugate at the intervals of two, three and five weeks;
(f) immunizing the birds thereafter with hapten-protein conjugate at five weeks intervals as long as the bird lays eggs;
(g) harvesting antibodies from the egg yolk of the birds.

2. A process as claimed in claim 1, wherein the desired hapten-protein conjugates having binding properties to DDT, Endosulphan and Hexachlorohexane.

3. A process as claimed in claim 1, wherein the production of hapten-protein conjugate, namely DDT-OH binding to DDT, as used in step (c) of claim 1, is as follows:
(a) succinylating 2,2,-Bis(4-chlorophenyl)-1,1,1-trichloroethanol overnight, using excess succinic anhydride in pyridine to obtain N-hydroxy succunimide;
(b) reacting N-hydroxy succinimide 183.5 mg., 0.5 mmol in dichloromethane in the presence of dicyclohexylurea and dimethylaminopyridine catalyst in the ratio 1:1:1:1.2 (hapten:NHS:DCC:DMAP) to convert into N-Hydroxy succinimide active ester; and
(c) obtaining active ester of DDT-OH hapten for use in conjugation by isolating dicyclohexylurea and evaporating dichloromethane.

4. A process as claimed in claim 1, wherein the production of hapten-protein conjugate namely octachloro cyclic hapten binding to Endosulphan as used in step(c) of claim 1, is as follows:
(a) dissolving about 3.73 g Heptachlor in 0.1 mol glacial acetic acid by warming;
(b) dissolving 1.085 g Tert-Butyl hypochlorite, in 0.1 mmol glacial acetic acid and adding to the first solution as obtained in step(a);
(c) refluxing the mixture on a water-bath for 1 hour;
(d) separating fine crystals of acetyl-chloro derivative of heptachlor;
(e) washing the crystals with acetone and drying with air;
(f) obtaining the crystalline product in a yield of about 3.02 g, m.p. 238 C. 1.09 g
(g) treating the product to get the pre-hapten 1,3,4,5,6,7,8,8-Octachloro-2-hydroxy-4, 7-methano-3a, and 4,7,7a-tetrahydroindane;
(h) dissolving the pre hapten in dichloromethane by adding N-hydroxysuccinimide and cooling the mixture to 0° C.;
(i) adding dicyclohexylcarbodiimide followed by dimethylaminopyridine;
(j) stirring the mixture overnight; and
(k) filtering off dichloromethane and evaporating dichloromethane to obtain the active ester of endosulphan.

5. A process as claimed in claim 1, wherein the production of conjugate hapten 2,4,5-Trichloro phenoxy acetic acid β-alanine (TCB) hapten binding to Hexachloro hexane, as per step(c), is as follows:
(a) adding of β-alanine spacer arm to 2,4,5 Trichlorophenoxyacetic acid by suspending 10 mM, 2.55 g of 2,4,5 Trichlorophenoxyacetic acid in 5.95 ml thionyl chloride (9 50 mmol);
(b) refluxing for 1 hour and removing unreacted thionyl chloride by evaporation;
(c) stirring the product with β-alanine (9 mmol, 0.66 g in 7.4 ml of 1M NaOH) at 0° C.;
(d) warming the product for over 16 hours at room temperature;
(e) isolating the resulting acid by acidification;
(f) partitioning into ethyl acetate;
(g) washing with water and brine;
(h) giving an yield of crude product hapten containing 2,4,5-Trichlorophenoxyacetic acid (2,4,5-T) as impurity;
(i) dissolving the impurity in acetone to obtain colorless flakes of the Trichlorobenzene(TCB) hapten;
(j) filtering and washing the colourless TCB hapten with acetone and drying in air;
(k) using silica gel precoated aluminum plates and a mixture of chloroform and methanol in a ratio of 85:15 as eluent showed a single spot in TLC analysis Rf-0.45 detected by spraying with 2% o-tolidine in acetone and exposure to Uv light/sunlight, at a melting range of 169-70° C.
(l) synthesizing the active ester of hapten 2,4,5-T-β-alanine at melting range of 102-104° C. by dissolving in dichloromethane.
(m) adding N-hydroxysuccinimide and the mixture is cooled in an ice-bath;
(n) adding Dimethylsulphoxide(DMSO) dropwise to the mixture until the hapten is dissolved;
(o) adding Dicyclohexylcarbodiimide to the mixture followed by adding dimethylaminopyridine catalyst;
(p) stirring the mixture overnight and the temperature slowly raised to the room temperature;
(q) filtering and evaporating acetone; and
(r) separating the active ester as a colorless solid.

6. A process as claimed in claim 1, wherein harvesting of antibodies as defined in step(g) of claim 1, is as follows:
(a) obtaining eggyolk without rupturing the yolk;
(b) adding 100 ml of Tris buffer saline for every 10 ml of yolk;
(c) removing the precipitate by centrifugation;
(d) adding to the supernatant the precipitating solution of magnesium chloride and phosphotungstic acid for centrifuging;
(e) discarding the pellet;
(f) adding to the supernatant a water soluble protein fraction 12% polyethylene glycol;
(g) incubating for 10 minutes and then centrifuging again;
(h) precipitating out the antibody;
(i) adding 10 ml of 10 mM phosphate buffer to dissolve the precipitate;
(j) cooling the antibody solution 0° Cl;
(k) adding 10 ml of pre-cooled ethanol;
(l) centrifuging the solution at 4° C. and dissolving the sediment in 10 mM phosphate buffer; and
(m) dialyzing against phosphate buffer for 24 h at 4° C. to obtain the yield of antibodies.

7. A process as claimed in claim 1, wherein harvesting of antibodies as defined in step(g) of claim 1, can also be conducted as follows:
(a) obtaining the egg yolk from the eggshell without rupturing the yolk membrane;
(b) adding for every 10 ml of yolk, 10 ml of distilled water;
(c) adding about 0.15% of kappa-carragenanin and left to stir for 30 minutes at room temperature;
(d) filtering and centrifuging the solution at for 15 minutes;
(e) passing through the DEAE-sephacel column prepared with 20 mM phosphate buffer pH 8.0;
(f) eluting with 0.2 M phosphate buffer pH 8.0;
(g) collecting the eluate and the absorbance read at 280 nm; and
(h) pooling and storing the peak fractions containing the antibody at 4° C.

8. A process as claimed in claim 6, wherein the lipid from egg yolk is precipitated out twice using the precipitating solution of phosphotungstic acid and magnesium chloride and centrifuged obtaining the antibody yield up to 75% from supernatant.

9. A process as claimed in claim 6, wherein pH of the water soluble protein fraction obtained after the removal of the lipids is adjusted to pH 5.0 to further precipitate out the antibodies for obtaining a yield of 80-90%.

10. A process as claimed in claim 7, wherein the yield of antibody is to the extent of 73%.

11. A process as claimed in claim 1, wherein the hyper immune eggs are collected daily and stored 40° C. until further use.

12. A process as claimed in claim 1, wherein commencing the production of the antibody from 7^{th} day after the immunization and continued for 60 days.

13. A process as claimed in claim 1, wherein the titer of the antibody produced is 165-225 mg/ml.

14. A process as claimed in claim 1, wherein production of the egg yolk antibody is more/equally sensitive to the polyclonal/monoclonal antibodies produced using mammals.

15. A process as claimed in claim 1, wherein production of the egg yolk antibodies relates to small molecules of pesticides.
